(19) United States
(12) Patent Application Publication
(10) Pub. No.: US 2026/0327565 A1
(43) Pub. Date: Oct. 22, 2026

(54) CRYOPRESERVED INTERMEDIATE AND POTENCY ASSAY FOR SAME

(71) Applicant: MESOBLAST INTERNATIONAL SARL, Meyrin (CH)

(72) Inventors: Silviu ITESCU, Melbourne (AU); Paul SIMMONS, Melbourne (AU)

(21) Appl. No.: 18/880,992

(22) PCT Filed: Jul. 5, 2023
(86) PCT No.: PCT/IB2023/056939

§ 371 Date: Jan. 3, 2025

(30) Foreign Application Priority Data
Jul. 5, 2022 (AU) 2022901890

(51) Int. Cl. C12N 5/0775; A61K 35/28; A61P 29/00; A61P 37/06; C12N 5/00; G01N 33/50

(52) U.S. Cl. CPC C12N 5/0668; A61K 35/28; A61P 29/00; A61P 37/06; C12N 5/562; G01N 33/5073; G01N 2333/7155

(57) ABSTRACT

The present disclosure relates to improved cellular intermediates and potency assays for obtaining the same. Such intermediates and assays may be suitable for use in providing cellular compositions for treating various inflammatory disorders.

### FIELD OF THE DISCLOSURE

The present disclosure relates to cellular intermediates, potency assays for characterizing the same and, selective methods for culture expansion. Such intermediates, assays and methods may be suitable for use in providing cellular compositions for treating various inflammatory disorders.

### BACKGROUND

Several cellular therapy products for regenerative or immune therapy applications have advanced to clinical evaluation and market authorization. However, release of these cellular therapy products onto the market is hindered by their complexity and heterogeneity, which makes identification of relevant biologic activities and thus, definition of consistent cellular therapy product quality difficult.

Physiochemical parameters (for example, characterization of size, morphology, light-scattering properties, tensile strength, cell number, confluence, identification of phenotypic markers, secreted substances, genotype, gene expression profile) are routinely used for identification and quantification of the active substance, intermediates, impurities and contaminants. However, physiochemical parameters cannot confirm that a product will be biologically active and potent (i.e., elicit the desired effect). In contrast, biologic characterization takes into account the effect of the product on biologic systems, either modelled in vitro or in vivo in animals and ultimately in the clinic.

Potency testing must demonstrate the relevant biologic activity or activities of the product. It is not a requirement for potency testing to reflect all of the product's biological functions, but it should indicate one or more relevant biological functions. It is expected that accuracy, sensitivity, specificity and reproducibility will be established for the analytic methods used in potency testing and that they be suitably robust.

There is a need to develop cell products for treatment of diseases, for example, where immunosuppression is desired. It is also preferable to identify parameters that are critical to the efficacy of cellular therapy products and to control them (e.g., via potency testing) such that products of consistent quality can be manufactured.

### SUMMARY OF THE DISCLOSURE

Previous potency testing has focused on characterizing the final cellular product for administration. While such testing remains important, those of skill in the art will appreciate that final cellular product is culture expanded from cryopreserved intermediates, also referred to as donor cell banks. Studies by the present inventors have surprisingly identified the importance of subjecting such cellular intermediates to potency testing prior to their culture expansion to final cellular product. For example, the present inventors have identified certain characteristics of donor cell banks which, when met, provide a “memory” for subsequent final product derived therefrom. Indeed, after controlling for downstream culture expansion processes, the present inventors surprisingly found that certain characteristic(s) of donor cell banks where associated with final cellular product that could impart improved 100 day survival in the context of treating the inflammatory disease GvHD. These findings represent a significant advance in the art as only therapeutically effective intermediate cell populations are selected for large scale expansion in 3D culture. Absent these findings, large scale expansion efforts may be wasted in the event that the resulting cell product does not meet the necessary therapeutic criteria for administration.

Accordingly, in certain embodiments, methods of the disclosure facilitate selection of the most appropriate cellular intermediates for culture expansion. In a first example, the present disclosure relates to a method of selecting a cell population for culture expansion, the method comprising: (i) obtaining a population of mesenchymal precursor lineage or stem cells (MLPSCs); (ii) culturing the MLPSC population in a culture medium; (iii) determining under culture conditions the level of IL2-RA inhibition; and, (iv) selecting an MLPSC population for further expansion that inhibits IL2-RA by at least 55% under culture conditions. In another example, the method comprises selecting an MLPSC population for further expansion that inhibits IL2-RA by at least 56% under culture conditions. In another example, the method comprises selecting an MLPSC population for further expansion that inhibits IL2-RA by at least 60% under culture conditions. In another example, the method comprises selecting an MLPSC population for further expansion that inhibits IL2-RA by at least 70% under culture conditions. In another example, the method comprises selecting an MLPSC population for further expansion that inhibits IL2-RA between 55% and 75% under culture conditions. In another example, the method comprises selecting an MLPSC population for further expansion that inhibits IL2-RA between 60% and 75% under culture conditions. In another example, the MLPSC population that is selected for further expansion also expresses at least 80 pg/ml of TNF-R1 under culture conditions. In another example, the MLPSC population that is selected for further expansion also expresses at least 100 pg/ml or at least 105 pg/ml TNF-R1 under culture conditions.

In an example, the further expansion occurs in 2D culture. In an example, the further expansion occurs in a cell factory. In another example, the further expansion occurs in 3D culture. In an example, the further expansion occurs in a bioreactor.

In an example, the method further comprises culture expanding a selected MLPSC population to provide a pharmaceutical composition. In an example, culture expanding the selected MLPSC population involves at least 3 to 5 passages.

In an example, culture expansion of a selected MLPSC population provides a MLPSC population characterized by IL2-RA inhibition greater than 60% under culture conditions. In another example, culture expansion of the selected MLPSC population provides a MLPSC population characterized by IL2-RA inhibition greater than 70% under culture conditions. In another example, culture expansion of the selected MLPSC population provides a MLPSC population characterized by IL2-RA inhibition greater than 75% under culture conditions. In another example, culture expansion of the selected MLPSC population provides a MLPSC population characterized by IL2-RA inhibition between 60 and 80% under culture conditions.

The findings of the present inventors also revealed that administration of cellular compositions culture expanded from cellular intermediates selected according to methods of the disclosure surprisingly increased 100 day survival in patients with inflammatory disease such as GvHD. In the context of GvHD increased 100 day survival was particularly pronounced in patients with severe disease, Grade D GvHD. Accordingly, in another embodiment, the present disclosure encompasses a method of determining therapeutic efficacy of an intermediate population of mesenchymal precursor lineage or stem cells (MLPSCs), the method comprising: (i) obtaining a population of MLPSCs; (ii) culturing the cells in a culture medium; and (iii) determining under culture conditions the level of IL2-RA inhibition, wherein at least 55% IL2-RA inhibition under culture conditions is indicative of therapeutic efficacy. In an example, IL2-RA inhibition greater than 56% under culture conditions is indicative of therapeutic efficacy. In an example, IL2-RA inhibition greater than 60% under culture conditions is indicative of therapeutic efficacy. In an example, IL2-RA inhibition greater than 70% under culture conditions is indicative of therapeutic efficacy. In another example, IL2-RA inhibition greater than 75% under culture conditions is indicative of therapeutic efficacy. In another example, IL2-RA inhibition between 55% and 75% under culture conditions is indicative of therapeutic efficacy. In another example, IL2-RA inhibition greater than 75% under culture conditions is indicative of therapeutic efficacy. In another example, IL2-RA inhibition between 60% and 75% under culture conditions is indicative of therapeutic efficacy.

In another example, the method of determining therapeutic efficacy further comprises determining under culture conditions the level of TNF-R1 expression under culture conditions. In an example, expression of at least 100 pg/ml TNF-R1 under culture conditions is indicative of therapeutic efficacy. In an example, expression of at least 105 pg/ml TNF-R1 under culture conditions is indicative of therapeutic efficacy. It will be appreciated that, in certain examples, after determining therapeutic efficacy, an intermediate population of MLPSCs may be culture expanded to provide a pharmaceutical composition. In such examples, the pharmaceutical composition may comprise a MLPSC population characterized by IL2-RA inhibition greater than 70% under culture conditions. In another example, the pharmaceutical composition may comprise a MLPSC population characterized by IL2-RA inhibition greater than 75% under culture conditions. In another example, the pharmaceutical composition may comprise a MLPSC population characterized by IL2-RA inhibition between 60% and 75% under culture conditions.

The present inventors findings also support new treatment for inflammatory disease. Accordingly, in an embodiment, the present disclosure encompasses a method of treating a subject with an inflammatory disease, the method comprising administering to a subject in need thereof a composition comprising a culture expanded population of mesenchymal lineage precursor or stem cells (MLPSCs), wherein the MLPSCs are culture expanded from a population of MLPSCs which inhibit IL2-RA by at least 55% under culture conditions. In an example, the culture expanded MLPSCs which comprise the administered composition inhibit IL2-RA by at least 56% under culture conditions. In an example, the culture expanded MLPSCs which comprise the administered composition inhibit IL2-RA by at least 60% under culture conditions. In an example, the culture expanded MLPSCs which comprise the administered composition inhibit IL2-RA by at least 70% under culture conditions. In another example, the culture expanded MLPSCs which comprise the administered composition inhibit IL2-RA by at least 75% under culture conditions. In another example, the culture expanded MLPSCs which comprise the administered composition inhibit IL2-RA by between 55% and 75% under culture conditions. In another example, the culture expanded MLPSCs which comprise the administered composition inhibit IL2-RA by between 55% and 75% under culture conditions. In another example, the culture expanded MLPSCs which comprise the administered composition inhibit IL2-RA by between 60% and 75% under culture conditions. In another example, the MLPSCs are culture expanded from a population of MLPSCs that also express at least 80 pg/ml TNF-R1 under culture conditions. In another example, the MLPSCs are culture expanded from a population of MLPSCs that also express at least 105 pg/ml TNF-R1 under culture conditions.

In another embodiment the present disclosure also relates to new cryopreserved cellular intermediates. Such intermediates are generally only passaged between 2 to 5 times following isolation before being cryopreserved. Accordingly, in an example, the present disclosure relates to a cryopreserved cellular intermediate comprising a population of culture expanded mesenchymal lineage precursor or stem cells (MLPSC), wherein the population of MLPSCs are culture expanded between 2 and 5 passages and the culture expanded MLPSCs: a) inhibit IL2-RA by at least 55% under culture conditions; and/or b) express at least 80 pg/ml TNF-R1 under culture conditions. In an example, the cellular intermediate: a) inhibits IL2-RA by at least 55% under culture conditions; and/or b) expresses at least 100 pg/ml TNF-R1 under culture conditions. In an example, the cellular intermediate: a) inhibits IL2-RA by at least 60% under culture conditions; and/or b) expresses at least 80 pg/ml TNF-R1 under culture conditions. In an example, the cellular intermediate: a) inhibits IL2-RA by at least 60% under culture conditions; and/or b) expresses at least 100 pg/ml TNF-R1 or at least 105 pg/ml TNF-R1 under culture conditions. In an example, the cellular intermediate: a) inhibits IL2-RA by at least 70% or at least 75% under culture conditions; and/or b) expresses at least 100 pg/ml TNF-R1 or at least 105 pg/ml TNF-R1 under culture conditions. In another example, the cellular intermediate: a) inhibits IL2-RA by between 55% and 75% under culture conditions; and/or b) expresses between 80 pg/ml and 105 pg/ml TNF-R1 under culture conditions.

In the above referenced examples, IL2-RA inhibition is measured by co-culturing the MLPCSs with activated PBMC.

In an example, cellular intermediates of the disclosure are culture expanded in 2D culture. For example, 2D culture may comprise cell culture in multilayer cell factories. In an example, cellular intermediates of the disclosure are culture expanded in around 5% CO₂, for example, 5%+/−2% CO₂. In an example, cell culture is performed with passive CO₂ priming. For example, cellular intermediates of the disclosure may be culture expanded in around 5% CO₂, for example, 5%+/−2% CO₂, with passive priming. For the avoidance of doubt, cell culture according to such examples does not comprise active CO₂ priming.

In an example, an inflammatory disease treated according to the present disclosure is graft versus host disease (GvHD). In an example, the GvHD is classified as Grade D. In an example, the GvHD is classified as chronic GvHD. In an example, subjects treated according to the present disclosure are refractory to steroid, immunosuppressant and/or a biologic therapy. In an example, the subject is refractory to a single biologic. In an example, the subject is refractory to multiple biologics.

In an example, treatment according to the disclosure increases 100 day survival to greater than 60%. In another example, treatment increases 100 day survival to greater than 70%. In another example, treatment according to the disclosure increases 100 day survival between 60 and 80%.

In an example, subjects treated according to the disclosure receive at least two doses of a composition disclosed herein.

In another example, the present disclosure relates to a method of manufacturing drug product which comprises a population of mesenchymal lineage precursor or stem cells (MLPSCs). In an example, the method comprises: acquiring a determination of whether a test population of MLPSCs have a predetermined level of IL2-RA inhibition under culture conditions, and culture expanding at least a portion of the test population of MLPSCs to a drug product if the test population of MLPSCs have at least the predetermined level of IL2-RA inhibition under culture conditions, thereby manufacturing the drug product; or discarding at least a portion of the test population of MLPSCs if the population of MLPSCs has less than the predetermined level of IL2-RA inhibition under culture conditions. In an example, the predetermined level of IL2-RA inhibition is at least 55% under culture conditions. In an example, the predetermined level of IL2-RA inhibition is at least 60% under culture conditions. In an example, the predetermined level of IL2-RA inhibition is at least 70% under culture conditions. In another example, the method further comprises acquiring a determination of whether a test population of MLPSCs have a predetermined level of TNF-R1 under culture conditions, and culture expanding at least a portion of the test population of MLPSCs to a drug product if the test population of MLPSCs have at least the predetermined level of IL2-RA inhibition under culture conditions and the predetermined level of TNF-R1 under culture conditions, thereby manufacturing the drug product; or discarding at least a portion of the test population of MLPSCs if the population of MLPSCs has less than the predetermined level of IL2-RA inhibition under culture conditions and less than the predetermined level of TNF-R1 under culture conditions. In an example, the predetermined level of TNF-R1 is at least 80 pg/ml. In another example, the predetermined level of TNF-R1 is at least 100 pg/ml. In another example, the predetermined level of TNF-R1 is at least 105 pg/ml.

In an example, expanding at least a portion of the test population of MLPSCs to a drug product provides a MLPSC population characterized by IL2-RA inhibition greater than 70% under culture conditions. In another example, expanding at least a portion of the test population of MLPSCs to a drug product provides a MLPSC population characterized by IL2-RA inhibition greater than 75% under culture conditions.

In an example, the MLPSCs are mesenchymal stem cells. In another example, the MLPSCs are mesenchymal precursor cells.

In an example, a composition disclosed herein is administered intravenously.

In an example, the methods of the disclosure encompass administering between 1×10⁷ and 2×10⁸ cells. For example, multiple doses of between 1×10⁷ and 2×10⁸ cells may be administered at regular intervals, for example, on days 0, 30, 60 and 90. In an example, the methods of the disclosure encompass administering about 1×10⁸ cells per dose.

In an example, a subject treated according to the disclosure receives a second dose within 7 days of being administered a first dose. In an example, the second dose is administered 4 days after the first dose. In an example, a dose comprises 2×10⁶ cells/kg of body weight.

In another example, compositions of the disclosure further comprise Plasma-Lyte A, dimethyl sulfoxide (DMSO), human serum albumin (HSA). In an example, the composition further comprises Plasma-Lyte A (70%), DMSO (10%), HSA (25%) solution, the HSA solution comprising 5% HSA and 15% buffer.

In an example, compositions of the disclosure comprise greater than 6.68×10⁶ viable cells/mL.

### BRIEF DESCRIPTION OF ACCOMPANYING FIGURES

FIG. 1: Changes in donor cell bank (DCB) manufacturing increased DCB potency as measured by % IL2-RA inhibition.

FIG. 2: Changes in donor cell bank (DCB) manufacturing increased TNF-R1 expression.

FIG. 3: Survival significantly improved in aGVHD patients receiving stem cell product made from DCB2 compared with stem cell product made from DCB1.

FIG. 4: Controlling for downstream culture expansion of DCB1 and DCB2, survival remains significantly improved in aGVHD patients receiving stem cell product made from DCB2 compared with stem cell product made from DCB1.

FIG. 5: Controlling for downstream culture expansion of DCB1 and DCB2, survival benefit further improved in grade D aGVHD patients receiving stem cell product made from DCB2 compared with stem cell product made from DCB1.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell therapy, immunology, immunohistochemistry, protein chemistry, and biochemistry).

Unless otherwise indicated, the surgical techniques utilized in the present disclosure are standard procedures, well known to those skilled in the art.

Methods of obtaining and enriching a population of mesenchymal lineage stem or precursor cells are known in the art. For example, enriched populations of mesenchymal lineage stem or precursor cells can be obtained by the use of flow cytometry and cell sorting procedures based on the use of cell surface markers that are expressed on mesenchymal lineage stem or precursor cells.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Selected Definitions

The term “intermediate” is used in the context of the present disclosure to refer to an intermediate population of MLPSCs that have been freshly isolated and subject to early stage culture expansion (e.g. around 2 passages). For example, production of the intermediate population of MLPSCs can include the isolation of nucleated bone marrow cells (NBMC) from Bone Marrow Aspirate (BMA), cell culture and two passages to expand the relevant cells. In an example, cells are then harvested and formulated for analysis according to the methods described herein. In an example the harvested cells are cryopreserved. Accordingly, production of an intermediate disclosed herein, is the first stage of the process of producing a composition for administration to a subject. While the above referenced example refers to isolation of NBMC from BMA, those of skill in the art will appreciate that relevant cells can be obtained from other suitable source(s) as discussed herein (e.g. adipose tissue) and subjected to the same early stage culture expansion to provide an intermediate encompassed by the present disclosure.

In certain examples disclosed herein, intermediates of the disclosure are culture expanded to provide a population of MLPSCs for administration. In an example, such populations can be referred to as a pharmaceutical composition or drug product (DP). For example, culture expansion of the intermediate involves cell culture and more passages (e.g. three more passages) for culture expansion. Culture expansion of intermediates is discussed further below.

Cryopreserved intermediates according to the present disclosure may also be referred to as a donor cell bank (DCB).

The terms “level” and “amount” are used to define the amount of a particular substance in a cell preparation. For example, a particular concentration, weight, percentage (e.g. v/v %) or ratio can be used to define the level of a particular substance. In an example, the level is expressed in terms of how much of a particular marker is expressed by cells disclosed herein under culture conditions. In an example, expression represents cell surface expression. In another example, the level is expressed in terms of how much of a particular marker is released from cells described herein into their culture medium under culture conditions.

In an example, the level is expressed in pg/ml. In another example, the level is expressed in pg per 10⁶ cells. The level of pg/ml can be converted to pg per 10⁶ cells if required. For example, in the context of TNF-R1, in an example, 80 pg/ml TNF-R1 corresponds to about 9.4 pg of TNF-R1 per 10⁶ cells. In an example, in the context of TNF-R1, in an example, 100 pg/ml TNF-R1 corresponds to about 11.5 pg of TNF-R1 per 10⁶ cells. In an example, 105 pg/ml TNF-R1 corresponds to about 12.5 pg of TNF-R1 per 10⁶ cells. In another example, 150 pg/ml TNF-R1 corresponds to about 17.5 pg of TNF-R1 per 10⁶ cells. In another example, 200 pg/ml TNF-R1 corresponds to about 23.5 pg TNF-R1 per 10⁶ cells and so on.

In an example, the level of a particular marker is determined under culture conditions. The term “culture conditions” is used to refer to cells growing in culture. In an example, culture conditions refers to an actively dividing population of cells. Such cells may, in an example, be in exponential growth phase. In an example, the cells may be in a stationary phase.

In an example, culture conditions encompass co-culture of an MLPSC population disclosed herein and a second cell population such as a population which comprises peripheral blood mononuclear cells (PBMC). In an example, co-culture comprises culturing an MLPSC population disclosed herein and a population of activated PBMC. For example, PBMC can be activated using anti-CD3 and anti-CD28 antibodies before co-culture with an MLSPC population disclosed herein.

In an example, “culture conditions” comprises co-culturing MLPSCs and T cells at a ratio of about 1 MLPSC:2 T cells, or less. For example, 1:3, 1:4, 1:5, 1:10, 1:20, 1:30, 1:40, 1:50, 1:60, 1:70 1:80, 1:90, or 1 MLPSC:100 T cells, or less. In this example, the level of IL2-RA inhibition is determined after about 30 to 84 hours of cell culture under culture conditions.

In an example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface of the cell may be measured by assessing a sample of cell lysate. In an example, the sample is taken when the cells are in exponential growth phase. In an example, the sample is taken after at least two or three days in culture. In another example, the sample is taken after about 30 to 84 hours of co-culture. In an example, the sample is taken from a co-culture of MLPSCs and activated PBMCs. In this example, the cell sample can be lysed and the level of a marker can be determined. For example, the level of IL2-RA may be determined using various methods such as an enzyme-linked immunosorbent assay (ELISA) based method. In an example, the ELISA comprises:
(i) adding sample diluent to each well of a microplate precoated with a monoclonal antibody specific for IL2-RA;
(ii) adding a co-cultured sample to a well of a microplate precoated with a monoclonal antibody specific for IL2-RA;
(iii) incubating the microplate for sufficient time to allow for the monoclonal antibody specific for IL2-RA to specifically bind to any IL2-RA in the sample;
(iv) washing the microplate;
(v) adding IL2-RA conjugate to the well;
(vi) incubating the microplate for sufficient time to allow the conjugate to specifically bind to any captured IL2-RA;
(vii) washing the microplate;
(viii) adding a substrate solution to the well;
(ix) incubating the microplate for sufficient time for colour development;
(x) adding a stop solution to the well;
(xi) reading optical density on a microplate reader set to 450 nm with wavelength correction at 570 nm;
(xii) determining the concentration of IL2-RA.

In another example, the level of IL2-RA is determined using fluorescence-activated cell sorting (FACS) using appropriate antibodies such as anti-CD25. Further antibodies may also be employed if required to distinguish CD25+ cell types.

In an example, the level of IL2-RA in co-culture is compared with the level of IL2-RA in cultured population of activated PBMC. Levels of IL2-RA are subsequently compared to provide a level of IL2-RA inhibition in co-culture. For example, the level of IL2-RA may be inhibited at least 60% in co-culture (i.e. MLPSC:activated PBMC) relative single culture (activated PBMC alone). Those of skill in the art will appreciate other suitable methods of determining level of IL2-RA under culture conditions.

Culture expanding cells from a cryopreserved intermediate means thawing cells subject to cryogenic freezing and in vitro culturing under conditions suitable for growth of the cells.

In an example, the “level” or “amount” of a particular marker such as IL2-RA or TNF-R1 is determined before cells have been cryopreserved. For example, the level may be determined after the first 2 to 5 passages of the cells. In another example, the level or amount of a particular marker is determined after a first cryopreservation of cells. In another example, the level is determined after a second cryopreservation of cells. For example, cells may be culture expanded to provide an intermediate, cryopreserved, defrosted before being re-seeded in culture so that the level of a particular marker can be determined under culture conditions and, cryopreserved a second time.

By “isolated” or “purified” it is meant a cell which has been separated from at least some components of its natural environment. This term includes gross physical separation of the cells from its natural environment (e.g. removal from a donor). The term “isolated” includes alteration of the cell's relationship with the neighboring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

The terms “passage”, “passaging” or “sub-culture” are used in the context of the present disclosure to refer to known cell culture techniques that are used to keep cells alive and growing under cultured conditions for extended periods of time so that cell numbers can continually increase. The degree of sub-culturing a cell line has undergone is often expressed as “passage number,” which is generally used to refer to the number of times cells have been sub-cultured. In an example, one passage comprises removing non-adherent cells and leaving adherent mesenchymal lineage precursor or stem cells. Such mesenchymal lineage precursor or stem cells can then be dissociated from the substrate or flask (e.g., by using a protease such as trypsin or collagenase), media can be added, optional washing (e.g., by centrifugation) may be performed, and then the mesenchymal lineage precursor or stem cells can be re-plated or reseeded to one or more culture vessels containing a greater surface area in total. The mesenchymal lineage precursor or stem cells can then continue to expand in culture. In another example, methods of removing non-adherent cells include steps of non-enzymatic treatment (e.g., with EDTA). In an example, mesenchymal lineage precursor or stem cells are passaged at or near confluence (e.g., about 75% to about 95% confluence). In an example, the mesenchymal lineage precursor or stem cells are seeded at a concentration of about 10%, about 15%, or about 20% cells/ml of culture medium.

The term “medium” or “media” as used in the context of the present disclosure, includes the components of the environment surrounding cells in culture. It is envisaged that the media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of inflammatory disease. In an example, treatment includes administering a population of culture expanded mesenchymal lineage stem or precursor cells disclosed herein. In an example, treatment response is determined relative to baseline. In an example, treatment response is determined relative to a control patient population.

In an example, methods of the present disclosure inhibit disease progression or disease complication in a subject. “Inhibition” of disease progression or disease complication in a subject means preventing or reducing the disease progression and/or disease complication in the subject. Accordingly, in an example, methods of the disclosure inhibit progression of disease severity. For example, such methods can inhibit progression of GvHD severity (i.e. inhibit progression in grading).

The term “prevent” or “preventing” as used herein include administering a population of mesenchymal lineage stem or precursor cells and/or progeny thereof and/or soluble factors derived therefrom to thereby stop or inhibit the development of at least one symptom of inflammatory disease.

The term “subject” as used herein refers to a human subject. For example, the subject can be an adult. Terms such as “subject”, “patient” or “individual” are terms that can, in context, be used interchangeably in the present disclosure.

As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding Angl would be considered genetically modified.

The term “total dose” is used in the context of the present disclosure to refer to the total number of cells received by the subject treated according to the present disclosure. In an example, the total dose consists of one administration of cells. In another example, the total dose consists of two administrations of cells. In another example, the total dose consists of three administrations of cells. In another example, the total dose consists of four or more administrations of cells. For example, the total dose can consist of two to four administrations of cells.

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death. Put another way, the end point increases survival. For example, 100 day survival may be increased when administering treatment according to the present disclosure.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, a composition of the disclosure is administered to a subject in an amount and for a time sufficient to induce an improvement, preferably a sustained improvement, in at least one indicator that reflects the severity of inflammatory disease. Various indicators that reflect the severity of the disease can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results. In an example, a clinically proven effective amount improves patient survival. In another example, a clinically proven effective amount reduces a subjects risk of mortality. In another example, a clinically proven effective amount increases 100 day survival. In an example, methods of the disclosure administer a clinically proven effective amount of a composition disclosed herein.

“Therapeutic efficacy” is used in the context of the present disclosure to refer to MLPSCs and compositions disclosed herein that can treat, inhibit and/or prevent disease. For example, therapeutically effective MLPSCs and compositions disclosed herein can treat, inhibit and/or prevent an inflammatory disease such as GvHD. In an example, therapeutically effective MLPSCs and compositions disclosed herein increase 100 day survival in subjects with GvHD.

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

### Inflammatory Disease

As used herein, the term, “inflammatory disease” should be taken to encompass diseases including but not limited to, graft versus host disease (GvHD), pruritus, skin inflammation, psoriasis, multiple sclerosis, rheumatoid arthritis, osteoarthritis, systemic lupus erythematosus, Hashimoto's thyroidis, myasthenia gravis, diabetes type I or II, diabetic nephropathy, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome, keratoconjunctivitis, uveitis, inflammatory bowel disease, Crohn's disease, ulcerative colitis, an inflammatory disease of the joints, skin, or muscle, acute or chronic idiopathic inflammatory arthritis, myositis, a demyelinating disease, chronic obstructive pulmonary disease, interstitial lung disease, interstitial nephritis and chronic active hepatitis.

In an example, the inflammatory disease is mediated by T-cell activation. Examples of such diseases include, GvHD, rheumatoid arthritis and diabetes. In an example, the inflammatory disease mediated by T-cell activation is refractory to at least one biologic therapy.

In an example, the inflammatory disease is hyperinflammation. The term “hyperinflammation” is used in the context of the present disclosure refer to severe and ongoing inflammatory process in body. For example, hyperinflammation can refer to severe and ongoing inflammatory process in airway and/or lungs, kidney or liver. In this way, hyperinflammation can affect multiple organs in the body and their vasculature. In an example, hyperinflammation is associated with a cytokine storm or cytokine release syndrome (CRS). In an example, the cytokine storm or CRS involves significant release of inflammatory cytokines such as IL-6.

In an example, the hyperinflammation leads to secondary (or acquired) hemophagocytic lymphohistiocytosis (sHLH). Accordingly, in an example, the methods of the present disclosure encompass treatment of hemophagocytic lymphohistiocytosis (HLH).

In an example, hyperinflammation is caused by a viral infection. For example, the hyperinflammation can be caused by a rhinovirus, an influenza virus, a respiratory syncytial virus (RSV) or a coronavirus. In an example, the hyperinflammation can be caused by a coronavirus. For example, the coronavirus can be coronavirus (SARS-CoV), Middle East Respiratory Syndrome coronavirus (MERS-CoV) or COVID-19. In an example, the hyperinflammation is caused by Epstein-Barr virus (EBV) or herpes simplex virus (HSV).

In an example, the inflammatory disease is multi-system inflammatory syndrome (MIS).

In an example, the inflammatory disease is ARDS. In an example, the ARDS is mild ARDS. In another example, the ARDS is moderate ARDS. In another example, the ARDS is severe ARDS. In another example, the ARDS is moderate or severe ARDS. In another example, the ARDS is moderate, severe or very severe ARDS.

In an example, the inflammatory disease is graft versus host disease (GvHD). “Graft versus Host Disease (GvHD)” is an immunological disorder that is the major factor that limits the success and availability of allogeneic bone marrow or stem cell transplantation. GvHD occurs in acute (aGvHD) or chronic (cGvHD) forms. Acute GvHD usually manifests within 100 days following bone marrow or stem cell transplantation. Chronic GvHD generally manifests later than aGvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain. In an example, patients with cGvHD have impaired reconstitution of CD5+B cells. In an example, cGvHD is refractory to steroid therapy. In an example, cGvHD is refractory to a biologic therapy. In an example cGvHD is refractory to steroid therapy and a biologic therapy.

GvHD severity can be graded by patterns of organ involvement and clinical performance status. Multi-organ involvement includes skin rash, liver involvement, and/or gastrointestinal (GI) involvement. Examples of skin rash, liver involvement, and GI involvement are provided in Table 1 and Table 2. In an example, the subject has GvHD with multi-organ involvement. In an example, subjects treated according to the disclosure have severe GvHD. In an example, severe GvHD is graded according to the Glucksberg scale (Glucksberg et al, 1974; Thomas et al, 1975) (Table 1). For example, the subject can have Grade II GvHD or Grade III/IV GvHD according to the Glucksberg scale. In one example, the subject has Grade II GvHD. In another example, the subject has Grade III/IV GvHD.

In another example, severe GvHD is graded according to IBMTR Severity Index (Table 2) (Rowlings et al., 1997). In one example, the subject has Grade B, Grade C, or Grade D GvHD according to the IBMTR severity scale. In an example, subject has Grade D GvHD.

In another example, the subject has Minnesota high risk GvHD. Minnesota high risk acute GvHD is defined as either skin stage 4; lower gastrointestinal (GI) stage 3-4 or liver stage 3-4; or skin stage 3+ and either lower GI 2-4 or liver stage 2-4 GvHD (MacMillan et al., 2015). In each of these examples, the subject can also have a high MAP score. For example, the subject can have a MAP score ≥0.29:

TABLE 1
Glucksberg clinical stage and grade of
acute GvHD (Rowlings et al., 1997).

| Stage | Skin | Liver | Intestinal tract |
|---|---|---|---|
| 1 | Maculopapular | Bilirubin 34-50 | >500 ml |
| | rash <25% of | μmol/l | diarrhoea/d |
| | body surface | | |
| 2 | Maculopapular | Bilirubin 51-102 | >1000 ml |
| | rash 25-50% of | μmol/l | diarrhoea/d |
| | body surface | | |
| 3 | Generalised | Bilirubin 103-225 | >1500 ml |
| | erythroderma | μmol/l | diarrhoea/d |
| 4 | Generalised | Bilirubin >225 | Severe abdominal |
| | erythroderma with | μmol/l | pain, with or |
| | bullous formation | | without ileus |
| | and desquamation | | |

| Grade | Degree of organ involvement |
|---|---|
| I | Stage 1-2 skin rash: no gut involvement: no liver involvement: |
| | no decrease in clinical performance |
| II | Stage 1-3 skin rash: stage 1 gut involvement or stage 1 liver |
| | involvement (or both): mild decrease in clinical performance |
| III | Stage 2-3 skin rash: stage 2-3 gut involvement or stage 2-4 liver |
| | involvement (or both): marked decrease in clinical performance |
| IV | Similar to Grade III with stage-4 organ involvement and |
| | extreme decrease in clinical performance |

TABLE 2
Criteria for IBMTR Severity Index for
acute GvHD (Rowlings et al., 1997).

| | | | | Liver | | | Gastrointestinal | |
|---|---|---|---|---|---|---|---|---|
| | Skin | | | involvement | | | invovlement | |
| | involvement | | | | Total | | | Volume |
| | | Extent | | | bili- | | | of |
| | Stage | of | | Stage | rubin | | Stage | diarrhoea |
| Index* | (max) | rash | | (max) | (μmol/l) | | (max) | (ml/d) |
| A | 1 | <25% | | 0 | <34 | | 0 | <500 |
| B | 2 | 25-50% | or | 1-2 | 34-102 | or | 1-2 | 550-1500 |
| C | 3 | >50% | or | 3 | 103-255 | or | 3 | >1500 |
| D | 4 | Bullae | or | 4 | >255 | or | 4 | Severe |
| | | | | | | | | pain and |
| | | | | | | | | ileus |

*Assign Index based on maximum in an individual organ system

In an example, subjects with severe GvHD do not respond to treatment with primary therapy. In an example, the subject has worsened within 3 days of primary therapy. In this example, the subject has worsened if their GvHD has increased in severity. For example, the subject's GvHD severity has increased according to MAP. In another example, the subject's GvHD severity has increased according to the Glucksberg scale. In another example, the subject's GvHD severity has increased according to the IMBTR scale. In another example, the subject's GvHD severity has increased according to organ involvement.

In another example, the subject has not responded within 7 days of a primary therapy. For example, the subject is refractory to primary therapy. In one example, primary therapy is systemic steroids. In one example, the subject has severe GvHD and is refractory to a steroid. In an example, the steroid is a corticosteroid. In another example, the steroid is a glucocorticoid. In another example, the steroid is prednisone. In another example, the subject has severe GvHD and is refractory to steroids and a second line therapy. For example, a second line therapy can include extracorporeal photopheresis, etanercept, infliximab, ruxolitinib, anti-thymocyte globulin, mycophenolate, alemtuzumab, basiliximab, or tocilizumab.

### Treatment Response

Methods of the present disclosure relate to the treatment of inflammatory diseases such as GvHD. As used herein, the terms “treating”, “treat”, “treatment”, “reducing progression” include administering a population of mesenchymal lineage stem or precursor cells and/or progeny thereof and/or soluble factors derived therefrom and/or extracellular vesicles derived therefrom to thereby reduce or eliminate at least one symptom of the inflammatory disease.

As used herein, the term “response” means response to therapy. In an example, a subject is considered to have had a response if they have an improvement in at least one organ without progression in any other organs and if additional therapy was not required. In another example, a subject is considered not to have had a response if they had stable or progressive GvHD or if the subsequent addition of secondary therapy is required. In this example, a subject who does not have a response is a non-responder.

In an example, treatment induces a partial response. In an example, the partial response is induced at least 28 after treatment is initiated. In an example, the partial response is induced 28 days after treatment is initiated. In an example, the partial response is induced at least 30 days after treatment is initiated. In an example, the partial response is induced at least 2 months after treatment is initiated. In another example, the partial response is induced at least 3 months after treatment is initiated. In another example, the partial response is induced within 3 months. In another example, the partial response is induced 28 to 56 days after treatment is initiated. In another example, the partial response is induced 100 days after treatment is initiated. In another example, the partial response is induced 160 days after treatment is initiated. In another example, the partial response is induced 180 days after treatment is initiated.

In another example, the partial response is induced after two doses. In another example, the partial response is induced after two doses administered once weekly. In another example, the partial response is induced after two doses administered once weekly every two weeks. In another example, the partial response is induced after three doses or more. In an example, in the context of GvHD, a partial response is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In an example, a partial response is characterized by a reduction in Skin % BSA score of at least one point. In another example, a partial response is characterized by a reduction in mouth score of at least one point. In another example, a partial response is characterized by a reduction in eye score of at least one point. In these examples, scores can be obtained using the NIH Consensus Criteria 2014 for GvHD.

In another example, a partial response is characterized by one or more or all of:
Reduction in Skin % BSA score of at least one point;
Reduction in mouth score of at least one point;
Reduction in eye score of at least one point.

There are various classification systems for characterizing GvHD (Lee, S., (2017) Blood., 129(1): 30-37). In an example, the NIH Consensus Criteria 2014 can be used for scoring outcomes disclosed herein (Jagasia et al., (2015) Biol Blood Marrow Transplant., 21:389-401). The components of the NIH Consensus Criteria 2014 are shown in the following table:

TABLE 3
Organ Scoring of GvHD

| Organ | Score 0 | Score 1 | Score 2 | Score 3 |
|---|---|---|---|---|
| Skin % BSA¹ | No BSA | 1-18% BSA | 19-50% BSA | >50% BSA |
| | involved | | | |
| Skin Features | No sclerotic | N/A | Superficial sclerotic | Deep sclerotic features; |
| | features | | features, but not | “hidebound”; impaired |
| | | | “hidebound” | mobility; ulceration |
| Mouth | No | Mild | Moderate symptoms | Severe symptoms with |
| | symptoms | symptoms | with disease signs with | disease signs with |
| | | with disease | partial limitation of | major limitation of oral |
| | | signs but not | oral intake | intake |
| | | limiting oral | | |
| | | intake | | |
| | | significantly | | |
| Eyes | No | Mild dry eye | Moderate dry eye | Severe dry eye |
| | symptoms | symptoms | symptoms partially | symptoms significantly |
| | | not affecting | affecting ADL | affecting ADL (special |
| | | ADL | (requiring lubricant | eyewear to relieve |
| | | (requirement | drops >3×/day or | pain) OR unable to |
| | | of lubricant | punctal plugs) | work because of ocular |
| | | drops ≤3×/day) | WITHOUT new | symptoms OR loss of |
| | | | vision impairment due | vision due to |
| | | | to keratoconjunctivitis | keratoconjunctivitis |
| | | | sicca (KCS) | sicca (KCS) |
| GI Tract | No | Symptoms | Symptoms associated | Symptoms associated |
| | symptoms | without | with mild to moderate | with significant weight |
| | | significant | weight loss (5-15%) | loss (>15%) within 3 |
| | | weight loss | within 3 months OR | months, requires |
| | | (<5%) | moderate diarrhea | nutritional supplement |
| | | | without significant | for most calorie needs |
| | | | interference with daily | OR esophageal dilation |
| | | | living | OR severe diarrhea |
| | | | | with significant |
| | | | | interference with daily |
| | | | | living |
| Liver | Normal total | Normal total | Elevated total bilirubin | Elevated total bilirubin |
| | bilirubin and | bilirubin with | but ≤3 mg/dL or | but >3 mg/dL |
| | ALT or | ALT ≥ 3 to | ALT > 5 × ULN | |
| | AP < 3 × ULN | 5 × ULN or | | |
| | | AP ≥ 3 × | | |
| | | ULN | | |
| Lungs Symptom | No | Mild | Moderate symptoms | Severe symptoms |
| Score | symptoms | symptoms | (SOB after walking on | (SOB at rests; requires |
| | | (SOB after | flat ground) | O2) |
| | | climbing one | | |
| | | flight of | | |
| | | steps) | | |
| Lungs Lung Score | FEV1 ≥80% | FEV1 60-79% | FEV1 40-59% | FEV1 ≤39% |
| Joints and Fascia | No | Mild | Tightness of arms or | Contractures WITH |
| | symptoms | tightness of | legs OR joint | significant decrease of |
| | | arms and | contractures, erythema | range of motion AND |
| | | legs, normal | thought to be due to | significant limitation of |
| | | or mild | fasciitis, moderate | ADL (unable to tie |
| | | decreased | decrease of range of | shoes, button shirts, |
| | | range of | motion AND mild to | dress shelf, etc.) |
| | | motion AND | moderate limitation of | |
| | | not affecting | ADL | |
| | | ADL | | |
| Genital Tract | No signs | Mild signs | Moderate signs and | Severe signs with or |
| | | and females | may have signs of | without symptoms |
| | | with or | discomfort on exam | |
| | | without | | |
| | | discomfort | | |
| | | on exam | | |
| Other features | No GvHD | Mild | Moderate | Severe |

In an example, a partial response is a decrease of ≥1 point on the organ-specific NIH Consensus Criteria 2014 score from the Table above. Accordingly, in an example, treatment induces ≥1 point decrease in Skin % BSA score. In another example, treatment induces ≥1 point decrease in mouth score. In another example, treatment induces ≥1 point decrease in eye score. In another example, treatment induces ≥1 point decrease in skin features score. In another example, treatment induces ≥1 point decrease in gastrointestinal tract score. In another example, treatment induces ≥1 point decrease in liver score. In another example, treatment induces ≥1 point decrease in lung symptom score. In another example, treatment induces ≥1 point decrease in lung FEV1 score. In another example, treatment induces ≥1 point decrease in joints and fascia score. In another example, treatment induces ≥1 point decrease in genital tract score.

In an example, the treatment induces a complete response after treatment is initiated. In an example, a complete response is the complete resolution of GvHD symptoms in all organs. In an example, the complete response is induced 28 days after treatment is initiated. In an example, the complete response is induced at least 28 after treatment is initiated. In an example, the complete response is induced at least 30 after treatment is initiated. In an example, the complete response is induced at least 2 months after treatment is initiated. In another example, the complete response is induced at least 3 months after treatment is initiated. In another example, the complete response is induced 28 to 56 days after treatment is initiated. In another example, the complete response is induced 100 days after treatment is initiated. In another example, the complete response is induced 160 days after treatment is initiated. In another example, the complete response is induced 180 days after treatment is initiated.

In another example, the complete response is induced after two doses. In another example, the complete response is induced after two doses administered once weekly. In another example, the complete response is induced after two doses administered once weekly every two weeks. In another example, the complete response is induced after three doses or more.

In another example, again in the context of GvHD, the treatment increases the probability of the subject's survival. For example, treatment increases the probability of the subject surviving for at least 20 days to 200 days after initiation of treatment. In one example, treatment increases the probability of the subject surviving for at least 180 days after initiation of treatment. In another example, treatment increases the probability of the subject surviving at least 100 days. In an example, the increased probability is determined relative to a subject that is not treated with a composition of the disclosure. In an example, treatment increases 100 day survival greater than 60%. In another example, treatment increases 100 day survival greater than 60%. In another example, treatment increases 100 day survival greater than 65%. In another example, treatment increases 100 day survival greater than 68%. In another example, treatment increases 100 day survival greater than 70%. In another example, treatment increases 100 day survival greater than 71%. In another example, treatment increases 100 day survival greater than 72%. In another example, treatment increases 100 day survival between 60% and 75%. In another example, treatment increases 100 day survival between 65% and 75%. In another example, treatment increases 100 day survival between 70% and 75%. In an example, 100 day survival is determined across a population of patients with the average 100 day survival observed in the population providing the measure of 100 day survival for each patient in the population. In an example, this measure is compared relative to a subject (or population of subjects) that is not treated with a composition of the disclosure.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (aMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-900), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

In an example, compositions of the disclosure comprise cells that induce new blood vessel formation in target tissue. In an example, the target tissue is the heart. In another example, the cells secrete factors that protect at risk or damaged myocardium. In an example, at risk or damaged myocardium has been subject to a lack of blood flow resulting from an ischemic event. In an example, the cells secrete factors that reduce apoptosis in cardiomyocytes.

### Culture Expansion of the Cells

In an example, mesenchymal lineage precursor or stem cells are culture expanded. “Culture expanded” mesenchymal lineage precursor or stem cells are distinguished from freshly isolated cells in that they have been cultured in cell culture medium and passaged (i.e. sub-cultured). In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population.

Accordingly, in an example, relevant cells are isolated and culture expanded for 2 passages to provide an intermediate MLPSC population. The intermediate MLPSC population is then culture expanded to provide a DP. For example, the intermediate cell population can be cultured for three more passages (i.e. 5 passages total) to provide a DP.

In an example, mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 7 passages. In these examples, MLPSCs may be culture expanded before being cryopreserved to provide an intermediate cryopreserved MLPSC population and then subject to further culture expansion.

In an example, the methods of the disclosure select an intermediate population (e.g. a cryopreserved intermediate) for further culture expansion based on certain criteria such as level of IL2-RA inhibition. For example, a cryopreserved intermediate can be selected for culture expansion if the MLPSC population inhibits IL2-RA by at least 55% under culture conditions. In another example, a cryopreserved intermediate can be selected for culture expansion if the MLPSC population inhibits IL2-RA by at least 60% under culture conditions.

In other examples, the methods of the disclosure encompass assessing therapeutic efficacy of a cryopreserved intermediate population of MLPSC.

In an example, DP compositions of the present disclosure are produced by culturing cells from an intermediate cryopreserved MLPSC population or, put another way, a cryopreserved intermediate.

In an example, compositions of the disclosure comprise mesenchymal lineage precursor or stem cells that are culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, mesenchymal lineage precursor or stem cells can be culture expanded for at least 3 passages. In an example, mesenchymal lineage precursor or stem cells can be culture expanded for at least 3-10 passages. In an example, mesenchymal lineage precursor or stem cells can be culture expanded for at least 3-8 passages. In an example, mesenchymal lineage precursor or stem cells can be culture expanded for at least 3-7 passages.

In an example, IL2-RA inhibition by mesenchymal lineage precursor or stem cells culture expanded from a cryopreserved intermediate disclosed herein are assessed under culture conditions (e.g. co-culture with T-cells). In an example, these MLPSCs inhibit IL2-RA greater than 70% under culture conditions. In another example, these MLPSCs inhibit IL2-RA greater than 72% under culture conditions. In another example, these MLPSCs inhibit IL2-RA between 70% and 80% under culture conditions.

In an example, culture expanded mesenchymal lineage precursor or stem cells can be culture expanded in medium free of animal proteins (e.g. in a xeno-free medium). In an example, mesenchymal lineage precursor or stem cells can be culture expanded in medium that is fetal bovine serum free.

In an embodiment, mesenchymal lineage precursor or stem cells can be obtained from a single donor, or multiple donors where the donor samples or mesenchymal lineage precursor or stem cells are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and, optionally
iv. thawing the cryopreserved in-process intermediate mesenchymal lineage precursor or stem cells preparation and expanding by passage expansion the in-process intermediate mesenchymal lineage precursor or stem cells preparation.

In the context of the present disclosure, certain assays may be performed between steps iii and iv. For example, IL2-RA inhibition may be determined under culture conditions after step iii. In an example, step iv is only performed if a desired level of IL2-RA inhibition is observed under culture conditions. In this example, the cell population is selected for culture expansion on the basis of IL2-RA inhibition under culture conditions.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell population is an intermediate population that is capable of inhibiting IL2-RA expression by CD3/CD28-activated PBMCs by at least 55% relative to a control. In another example, the expanded mesenchymal lineage precursor or stem cell population is an intermediate population that is capable of inhibiting IL2-RA expression by CD3/CD28-activated PBMCs by at least 60% relative to a control. In another example, the expanded mesenchymal lineage precursor or stem cell population is an intermediate population capable of inhibiting IL2-RA expression by CD3/CD28-activated PBMCs by at least about 65% relative to a control. In another example, the expanded mesenchymal lineage precursor or stem cell population is an intermediate population capable of inhibiting IL2-RA expression by CD3/CD28-activated PBMCs by at least about 70% relative to a control. In another example, the expanded mesenchymal lineage precursor or stem cell population is an intermediate population capable of inhibiting IL2-RA expression by CD3/CD28-activated PBMCs between 55 and 70% relative to a control. In another example, the expanded mesenchymal lineage precursor or stem cell population is an intermediate population capable of inhibiting IL2-RA expression by CD3/CD28-activated PBMCs between 60 and 70% relative to a control.

In an example, the expanded mesenchymal lineage precursor or stem cell population is expanded from an intermediate MLPSC population and is capable of inhibiting IL2-RA expression by CD3/CD28-activated PBMCs by between at least 70% relative to a control. In another example, the expanded mesenchymal lineage precursor or stem cell population is expanded from an intermediate MLPSC population and is capable of inhibiting IL2-RA expression by CD3/CD28-activated PBMCs between 70 and 75% relative to a control.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 2D culture. For example, mesenchymal lineage precursor or stem cells of the disclosure can be culture expanded in a cell factory. In certain examples, 3D culture of intermediates disclosed herein may follow using, for example, a bioreactor. In an example, mesenchymal lineage precursor or stem cells of the disclosure are initially culture expanded in 2D culture prior to being further expanded in 3D culture. In an example, intermediate cell populations of the disclosure have not been culture expanded in 3D culture. In an example, IL2-RA inhibition is assessed before subsequent culture expansion in a cell factory or 3D culture.

In an example, mesenchymal lineage precursor or stem cells of the disclosure are culture expanded from an intermediate population. In an example, mesenchymal lineage precursor or stem cells of the disclosure are culture expanded from the intermediate in 2D culture before seeding in 3D culture.

In the context of both intermediate populations and therapeutic compositions expanded from the same, in an example, mesenchymal lineage precursor or stem cells of the disclosure are culture expanded in 2D culture for at least 3 days before seeding in a further culture system such as cell factory or 3D culture in a bioreactor. In an example, mesenchymal lineage precursor or stem cells of the disclosure are culture expanded in 2D culture for at least 4 days before seeding in a further culture system. In an example, mesenchymal lineage precursor or stem cells of the disclosure are culture expanded in 2D culture for between 3 and 5 days before seeding in a further culture system. In these examples, 2D culture can be performed in a cell factory. Various cell factory products are available commercially (e.g. Thermofisher, Sigma, Corning). In an example, the cell factory has at least 5 layers. In an example, the cell factory has at least 10 layers. In an example, the cell factory has at least 20 layers. 3D culture may be performed in various bioreactor types such as stirred tank, wave bag, and vertical wheel.

In an example, CO₂ is provided during culture expansion of MLPSCs. In an example, MLPSCs are culture expanded in less than 9% CO₂. In an example, MLPSCs are culture expanded in less than 8% CO₂. In an example, MLPSCs are culture expanded in 5% CO₂. For example, MLPSCs can be culture expanded in 5%+/−2% CO₂. In an example, the MLPSCs are culture expanded with passive priming of CO₂. For example, cell factories can be passively primed with 5% CO₂.

Priming cell factories maintains the CO₂ tension between the cell factory and incubator and stabilizes the pH level of the growth medium. Active priming involves actively passing CO₂ gas through a bacterial vent air filter into each culture vessel (e.g. cell factory) for a defined period of time (e.g. around 10 minutes). However, active priming has the potential to introduce contamination into culture as it requires an open port to provide gas. Passive priming involves placing a closed culture system into an incubator at appropriate CO₂ concentration prior to cell seeding (e.g. around 12 to 72 hours).

In an example, cells of the disclosure are STRO-3+ before they are culture expanded to provide an intermediate cell population.

### Cell Culture Medium

Mesenchymal lineage precursor or stem cells disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. In other examples the culture medium is fetal bovine serum free culture medium comprising growth factors that promote mesenchymal lineage precursor or stem cell proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In an example, the FGF2 level is about 1 ng/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is about 10 ng/ml. In another example, the level of PDGF-BB is at least about 10 ng/ml or less.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 1 ng/ml and 8 ng/ml. In another example, the level of EGF is between about 3 ng/ml and 6 ng/ml. In another example, the level of EGF is about 5 ng/ml. In another example, the level of EGF is about 5 ng/ml or less.

In the above examples, basal medium such as Alpha MEM or StemSpan™ can be supplemented with the referenced quantity of growth factor. In an example, the culture medium comprises Alpha MEM or StemSpan™ supplemented with 10 ng/ml PDGF-BB, 5 ng/ml EGF and 1 ng/ml FGF.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure maintain stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

Co-Culture of Mesenchymal Lineage Precursor or Stem Cells with Stimulated or Activated T Cells

Interleukin-2 receptor alpha chain (“IL2-RA”; also called CD25) is a validated marker of T-cell activation. In certain embodiments methods of the disclosure measure the inhibition of T-cell activation. In an example, T-cell activation is determined based on the level of T-cell IL2-RA expression following co-culture of activated PBMC with mesenchymal lineage precursor or stem cells. Inhibition of IL2-RA expression is associated with a suppressive effect on T cell activation.

In an example, activated PBMC are co-cultured with mesenchymal lineage precursor or stem cells in a culture medium comprising at least one T cell stimulating agent, preferably at a concentration capable of stimulating and/or activating the T cells. In another embodiment, the T cells are first stimulated and/or activated prior to co-culture with the mesenchymal lineage precursor or stem cells.

In an example, mesenchymal lineage precursor or stem cells are co-cultured with PBMC in a culture medium comprising an agent that can stimulate CD3 and an agent that can stimulate CD28 on T cells, such as an antibody to CD3 and an antibody to CD28, for example, mouse anti-human CD3 and mouse anti-human CD28. In an example, the antibody to CD3 and/or the antibody to CD28 is added to the culture medium in soluble form, each at a concentration of about 2 μg/ml.

In an embodiment, the PBMC are co-cultured with mesenchymal precursor or stem cells at a ratio of 5 PBMC:1 mesenchymal precursor or stem cell. For example, 1×10⁶ PBMCs could be co-cultured with 2×10⁵ MLPSCs. In an example, cells can be co-cultured in a final volume of 1 ml.

### Determining Therapeutic Efficacy

In an embodiment, the present disclosure relates to methods of determining therapeutic efficacy of intermediate populations of mesenchymal precursor lineage or stem cells (MLPSCs). In certain examples, such methods may be useful for establishing a “memory” in an intermediate cell population for subsequent final product derived therefrom. In other words, such methods demonstrate therapeutic efficacy in the intermediate cell population and the resulting product(s) culture expanded therefrom. Therapeutic efficacy of resulting product (or selected product) may be characterized using additional assays in certain examples, if required.

In an example, the method for determining therapeutic efficacy of an intermediate population of mesenchymal precursor lineage or stem cells (MLPSCs) comprises: (i) obtaining a population of MLPSCs; (ii) culturing the cells in a culture medium; and (iii) determining under culture conditions the level of IL2-RA inhibition, wherein at least 55% IL2-RA inhibition under culture conditions is indicative of therapeutic efficacy. In another example, IL2-RA inhibition greater than 56% is indicative of therapeutic efficacy. In another example, IL2-RA inhibition greater than 60% is indicative of therapeutic efficacy. In another example, IL2-RA inhibition greater than 70% is indicative of therapeutic efficacy.

In other examples, methods of determining therapeutic efficacy further comprise determining under culture conditions the level of TNF-R1 expression, wherein expression of at least 100 pg/ml TNF-R1 is indicative of therapeutic efficacy. In another example, expression of at least 105 pg/ml TNF-R1 is indicative of therapeutic efficacy.

Performing the methods of determining therapeutic efficacy disclosed herein can provide suitable intermediates for culture expansion to therapeutically effective cell compositions. In an example, such culture expanded compositions are characterized by a threshold level of IL2-RA inhibition. In an example, the level of IL2-RA inhibition is greater than the level observed in the preceding intermediate population. In an example, the level of IL2-RA inhibition is greater than 70% under culture conditions. In another example, the level of IL2-RA inhibition is greater than 75% under culture conditions. In another example, the level of IL2-RA inhibition is between 65 and 75% under culture conditions. In another example, the level of IL2-RA inhibition is between 70 and 75% under culture conditions.

### Determining the Amount of IL2-RA Levels

The present disclosure contemplates any form of assay, including, for example, Western blot, enzyme-linked immunosorbent assay (ELISA), fluorescence-linked immunosorbent assay (FLISA), competition assay, radioimmunoassay, lateral flow immunoassay, flow-through immunoassay, electrochemiluminescent assay, nephelometric-based assays, turbidometric-based assay, fluorescence activated cell sorting (FACS)-based assays for determining the level of IL2-RA.

Following co-culture of MLPSCs and T cells, the cells can be collected and lysed using well-known methods in the art. The cell lysates can then be assayed for the presence of IL2-RA by, for example, ELISA or FLISA. Alternatively, the level of IL2-RA expression may be determined by assaying intact cells by, for example, flow cytometry.

In an example, the inhibition of IL2-RA expression is measured by comparing the level of IL2-RA expression of a population of cells comprising T cells to the level of IL2-RA of a population of cells following co-culture of a population of cells comprising T cells and a population of cells comprising mesenchymal lineage precursor or stem cells, and the difference expressed as “percentage inhibition”.

The assays described above are readily modified to use chemiluminescence or electrochemiluminescence as the basis for detection.

As will be apparent to the skilled person, other detection methods based on an immunosorbent assay are useful in the performance of the present disclosure. For example, an immunosorbent method based on the description above using a radiolabel for detection, or a gold label (e.g., colloidal gold) for detection, or a liposome, for example, encapsulating NAD+ for detection or an acridinium linked immunosorbent assay.

In some examples of the disclosure, the level of IL2-RA is determined using a surface plasmon resonance detector (e.g., BIAcore™, GE Healthcare, Piscataway, N.J.), a flow through device (e.g., as described in U.S. Pat. No. 7,205,159), a micro- or nano-immunoassay device (e.g., as described in U.S. Pat. No. 7,271,007), a lateral flow device (e.g., as described in US publication 20040228761 or US publication 20040265926), a fluorescence polarization immunoassay (FPIA, e.g., as described in U.S. Pat. No. 4,593,089 or U.S. Pat. No. 4,751,190), or an immunoturbidimetric assay (e.g., as described in U.S. Pat. No. 5,571,728 or U.S. Pat. No. 6,248,597).

### Determining the Amount of TNFR1 Levels

In certain examples, methods of the disclosure also include the step of determining expression of TNFR1 by the mesenchymal lineage precursor or stem cells.

The skilled person will appreciate that the methods described above for the detection of IL2-RA expression can also be employed to detect TNFR1 expression. In a preferred embodiment, cell lysates as assayed by ELISA or FLISA.

In one form, such an assay involves immobilizing a TNFR1 binding protein onto a solid matrix. A test sample is then brought into direct contact with the TNFR1 binding protein and TNFR1 in the sample is bound or captured. Following washing to remove any unbound protein in the sample, a protein that binds to TNFR1 at a distinct epitope is brought into direct contact with the captured TNFR1. This detector protein is generally labeled as described above. Alternatively, a second labeled protein can be used that binds to the detector protein. Following washing to remove any unbound protein the detectable reporter molecule is detected by the addition of a substrate in the case of an ELISA as described above. The level of TNFR1 in the sample is then determined using a standard curve that has been produced using known quantities of the marker or by comparison to a control sample.

### Selecting Cells for Culture Expansion

In an embodiment the present disclosure encompasses selecting cells for culture expansion based on certain criteria. In example, the cells comprise an intermediate MLPSC population. In an example, the MLPSC population is assessed for the requisite criteria and, when met, said population is selected for culture expansion.

In an example, an MLPSC population is selected for further expansion if it inhibits IL2-RA by at least 55% under culture conditions. In an example, the MLPSC population is an intermediate population. In an example, an intermediate MLPSC population is selected for further expansion if it inhibits IL2-RA by at least 55% under culture conditions. In another example, an intermediate MLPSC population is selected for further expansion if it inhibits IL2-RA by at least 56% under culture conditions. In another example, an intermediate MLPSC population is selected for further expansion if it inhibits IL2-RA by at least 60% under culture conditions. In another example, an intermediate MLPSC population is selected for further expansion if it inhibits IL2-RA by at least 70% under culture conditions. In another example, an intermediate MLPSC population is selected for further expansion if it inhibits IL2-RA by between 55 and 70% under culture conditions. In another example, an intermediate MLPSC population is selected for further expansion if it inhibits IL2-RA by between 60 and 70% under culture conditions. In another example, an intermediate MLPSC population selected for further expansion also expresses at least 80 pg/ml TNF-R1 under culture conditions. In another example, an intermediate MLPSC population selected for further expansion also expresses at least 105 pg/ml TNF-R1 under culture conditions. In another example, an intermediate MLPSC population selected for further expansion also expresses at least 120 pg/ml TNF-R1 under culture conditions. In another example, an intermediate MLPSC population selected for further expansion also expresses between 80 pg/ml TNF-R1 and 120 pg/ml TNFR1 under culture conditions.

Selection processes are not particularly limited so long as they are able to select cell populations characterized by the relevant criteria such as % IL2-RA inhibition. In an example, a series of intermediate MLPSC populations are assessed for IL2-RA inhibition under culture conditions and those populations which inhibit IL2-RA inhibition by at least 55% under culture conditions are selected for further expansion. In an example, a series of intermediate MLPSC populations are assessed for IL2-RA inhibition under culture conditions and those populations which inhibit IL2-RA inhibition by at least 60% under culture conditions are selected for further expansion. In an example, those intermediate MLPSC populations meeting the relevant threshold level of IL2-RA inhibition are pooled before further expansion.

It should be appreciated that the selection process does not require immediate culture expansion. Rather “selected” populations can be cryopreserved and culture expanded at a later stage. In an example, a fraction of the intermediate cell population is culture expanded with the remainder of the population being cryopreserved for culture expansion at a later stage.

In an example, selected cell populations are immediately culture expanded. In another example, selected cell populations are cryopreserved to allow culture expansion at a later stage.

In an example, a selected cell population is culture expanded to provide a pharmaceutical composition. In an example, the pharmaceutical composition is characterized by certain criteria such as level of IL2-RA inhibition. In an example, culture expansion of the selected MLPSC population provides an MLPSC population characterized by IL2-RA inhibition greater than 60%. In another example, culture expansion of the selected MLPSC population provides an MLPSC population characterized by IL2-RA inhibition greater than 70% under culture conditions. In another example, culture expansion of the selected MLPSC population provides an MLPSC population characterized by IL2-RA inhibition greater than 75% under culture conditions. In another example, culture expansion of the selected MLPSC population provides an MLPSC population characterized by IL2-RA inhibition between 60% and 80% under culture conditions.

### Drug Product and Methods of Manufacturing the Same

The present inventors have further identified a method for manufacturing drug product by selecting a population of MLPSCs for culture expansion based on predetermined level of IL2-RA inhibition under culture conditions. Accordingly, in an example, the disclosure provides a method of manufacturing drug product which comprises a population of MLPSCs, the method comprising: acquiring a determination of whether a test population of MLPSCs have a predetermined level of IL2-RA inhibition under culture conditions, and culture expanding at least a portion of the test population of MLPSCs to a drug product if the test population of MLPSCs have at least the predetermined level of IL2-RA inhibition under culture conditions, thereby manufacturing the drug product; or discarding at least a portion of the test population of MLPSCs if the population of MLPSCs has less than the predetermined level of IL2-RA inhibition under culture.

In an example, the method further comprises acquiring a determination of whether a test population of MLPSCs have a predetermined level of TNF-R1 under culture conditions, and culture expanding at least a portion of the test population of MLPSCs to a drug product if the test population of MLPSCs have at least the predetermined level of IL2-RA inhibition under culture conditions and the predetermined level of TNF-R1 under culture conditions, thereby manufacturing the drug product; or discarding at least a portion of the test population of MLPSCs if the population of MLPSCs has less than the predetermined level of IL2-RA inhibition under culture conditions and less than the predetermined level of TNF-R1 under culture conditions.

In an example, the test population is obtained from a population of MLPSCs in 3D culture. For example, the MLPSCs can be in a bioreactor culture. In an example, the test population is obtained from cryopreserved population of MLPSCs. In an example, the test population is representative of a larger population of MLPSCs such as multiple cryopreserved populations of MLPSCs.

As used herein, the term “predetermined level” refers to a level of a marker that indicates therapeutic efficacy of a culture expanded drug product. In an example, the predetermined level is a level of IL-2RA inhibition that indicates therapeutic efficacy of a culture expanded drug product. In an example, the predetermined level of IL-2RA inhibition is at least 55% under culture conditions. In an example, the predetermined level of IL-2RA inhibition is at least 56% under culture conditions. In an example, the predetermined level of IL-2RA inhibition is at least 60% under culture conditions. In another example, the predetermined level of IL-2RA inhibition is at least 70% under culture conditions. In an example, the predetermined level of IL-2RA inhibition is between 55% and 80% under culture conditions. In another example, the predetermined level of IL-2RA inhibition is between 55% and 75% under culture conditions. In another example, the predetermined level of IL-2RA inhibition is between 60% and 80% under culture conditions. In another example, the predetermined level of IL-2RA inhibition is between 60% and 75% under culture conditions.

In an example, the predetermined level is a level of TNF-R1 under culture conditions. In an example, the predetermined level of TNF-R1 is at least 80 pg/ml under culture conditions. In another example, the predetermined level of TNF-R1 is at least 100 pg/ml under culture conditions. In another example, the predetermined level of TNF-R1 is at least 105 pg/ml under culture conditions.

In an example, the predetermined level is a clinically proven effective predetermined level. In an example, the level is clinically proven effective in the treatment of an inflammatory disease such as GvHD. In another example, the predetermined level is predetermined by a regulatory authority such as the US Food and Drug Administration (FDA). In an example, the predetermined level corresponds with increased survival in patients with GvHD.

In an example, culture expanding at least a portion of the test population of MLPSCs to a drug product provides a MLPSC population characterized by IL2-RA inhibition greater than 70% culture conditions. In another example, culture expanding at least a portion of the test population of MLPSCs to a drug product provides a MLPSC population characterized by IL2-RA inhibition greater than 75% under culture conditions.

In an example, the present disclosure provides methods of manufacturing MLPSC drug product, such methods include a first step of providing (e.g., culture expanding (e.g., in small scale or large scale cell culture) or manufacturing) or obtaining (e.g., receiving and/or purchasing from a third party (including a contractually related third party or a non-contractually-related (e.g., an independent) third party) a test MLPSC population (e.g., a sample of a test MLPSC population), a second step of acquiring (e.g., detecting, measuring, receiving, or obtaining) at least one value for an MLPSC parameter listed in Table A for the test MLPSC population, and a third step of culture expanding at least a portion of the test MLPSC population (e.g., expanding a portion of a manufacturing lot, culture, or run, an entire manufacturing lot, culture, or run, or multiple manufacturing lots, cultures, or runs) to MLPSC drug product (e.g., in a form or packaging intended for administration as described subsequently herein; optionally cryopreserved) if the at least one value for the test MLPSC population meets a reference criterion shown in Table A for the parameter, thereby manufacturing MSC drug product. In an example, the value(s) comprise parameter number 1. In another example, the value(s) comprise parameter number 2. In another example, the value(s) comprise parameter number 1 and 2.

In an example, such methods comprise a second step which includes acquiring values for level of DP IL2-RA inhibition under culture conditions, and a third step of such methods includes releasing at least a portion (i.e. release criteria) of the DP if the values for the level of IL2-RA inhibition under culture conditions is greater than 55%. In another example, the third step of such methods includes releasing at least a portion of the DP if the values for the level of IL2-RA inhibition under culture conditions is greater than 56%. In another example, the third step of such methods includes releasing at least a portion of the DP if the values for the level of IL2-RA inhibition under culture conditions is greater than 60%. In another example, the third step of such methods includes releasing at least a portion of the DP if the values for the level of IL2-RA inhibition under culture conditions is greater than 65%. In another example, the third step of such methods includes releasing at least a portion of the DP if the values for the level of IL2-RA inhibition under culture conditions is greater than 70%. In another example, the third step of such methods includes releasing at least a portion of the DP if the values for the level of IL2-RA inhibition under culture conditions is greater than 75%.

TABLE A

| Parameter | | |
|---|---|---|
| # | Description | Reference Criterion |
| 1 | Level of IL2-RA inhibition | Between 55% and 80%; or, |
| | under culture conditions | Between 60% and 80% |
| 2 | Level of TNF-R1 under culture | ≥80 pg/ml. |
| | conditions | |

### Compositions of the Disclosure

In an example, the present disclosure encompasses intermediate MLPSC populations with a memory for subsequent final product derived from that intermediate. Such intermediates are distinguished from freshly isolated cells in that they have been culture expanded from a population of the same to provide an intermediate. They are also distinguished from a final composition for administration in that they need to be further culture expanded to provide such a composition. Accordingly, in an example, intermediate MLPSC populations of the disclosure have been passaged at least 2 or at least three times. In an example, the intermediate cell population is passaged between 1 and 4 times. In an example, the intermediate cell population is cryopreserved.

In an example, the intermediate cell population is characterized by a threshold level of IL2-RA inhibition. In another example, the intermediate cell population is characterized by a threshold level of TNF-R1 expression. Accordingly, in an example, the present disclosure relates to a cryopreserved cellular intermediate comprising a population of culture expanded mesenchymal lineage precursor or stem cells (MLPSC), wherein the population of MLPSCs are culture expanded between 2 and 5 passages and the culture expanded MLPSCs: a) inhibit IL2-RA by at least 55% under culture conditions; and/or b) express at least 80 pg/ml TNF-R1 under culture conditions. In an example, the MLPSCs in the cellular intermediate: a) inhibit IL2-RA by at least 60% under culture conditions; and/or b) expresses at least 80 pg/ml TNF-R1 under culture conditions. In an example, the MLPSCs in the cellular intermediate: a) inhibit IL2-RA by at least 70% under culture conditions; and/or b) expresses at least 100 pg/ml TNF-R1 under culture conditions. In an example, the MLPSCs express at least 105 pg/ml TNF-R1 under culture conditions.

In one example of the present disclosure the mesenchymal lineage precursor or stem cells and/or progeny thereof and/or soluble factor derived therefrom are provided and/or administered in the form of a composition. Such compositions are culture expanded from an intermediate MLPSC population disclosed herein. In one example, such a composition comprises a pharmaceutically acceptable carrier and/or excipient. Accordingly, in an example, compositions of the disclosure can comprise mesenchymal lineage precursor or stem cells which are culture expanded from an intermediate population disclosed herein. In an example, such compositions can be a pharmaceutical composition.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay metabolic syndrome and/or obesity.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). In an example, the composition comprises 2×10⁶ cells/kg. The exact amount of cells to be administered is dependent upon a variety of factors, including the age, weight, and sex of the subject, and the extent and severity of the disorder being treated.

In an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the methods of the present disclosure encompass administering a total dose of 600 million cells. For example, a subject treated according to the present disclosure can receive multiple doses of an above referenced composition so long as the total dose of cells does not exceed 600 million cells. For example, the subject may receive 3 doses of 200 million cells. In an example, the total dose of cells is 500 million cells. In an example, the total dose of cells is 400 million cells. For example, the subject may receive 4 doses of 100 million cells. In an example, the subject receives 1 dose of 100 million cells at baseline followed by three doses of 100 million cells administered one per month over three months. In an example, a dose is 2×10⁶ cells/kg. In an example, a dose is 2×10⁶ cells/kg and the subject receives 2 doses or 3 doses. In an example, a dose is 2×10⁶ cells/kg and the subject receives more than 3 doses.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte A™. 100 mL of PlasmaLyte A™ contains 526 mg of sodium chloride, USP (NaCl); 502 mg of sodium gluconate (C₆H₁₁NaO₇); 368 mg of sodium acetate trihydrate, USP (C₂H₃NaO₂·3H₂O); 37 mg of potassium chloride, USP (KCl); and 30 mg of magnesium chloride, USP (MgCl₂·6H₂O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze™/50% αMEM/7.5% DMSO and cooled in a controlled-rate freezer.

The cryopreserved composition may be thawed and administered directly to the subject or added to another solution, for example, comprising HA. Alternatively, the cryopreserved composition may be thawed and the mesenchymal lineage precursor or stem cells resuspended in an alternate carrier prior to administration.

In an example, cellular compositions of the disclosure can comprise Plasma-Lyte A, dimethyl sulfoxide (DMSO) and human serum albumin (HSA). For example, compositions of the disclosure may comprise Plasma-Lyte A (70%), DMSO (10%), HSA (25%) solution, the HSA solution comprising 5% HSA and 15% buffer.

In an example, the compositions described herein may be administered as a single dose.

In some examples, the compositions described herein may be administered over multiple doses. For example, at least 2, at least 3, at least 4 doses. In other examples, compositions described herein may be administered over at least 5, at least 6, at least 7, at least 8, at least 9, at least 10 doses.

In an example, mesenchymal lineage precursor or stem cells may be administered systemically. In another example, compositions of the disclosure are administered intravenously.

In an example, mesenchymal lineage precursor or stem cells are administered once weekly. For example, mesenchymal lineage precursor or stem cells can be administered once weekly every two weeks. In another example, mesenchymal lineage precursor or stem cells are administered twice weekly. In an example, mesenchymal lineage precursor or stem cells can be administered once monthly. In an example, two doses of mesenchymal lineage precursor or stem cells are administered once weekly over two weeks. In another example, two doses of mesenchymal lineage precursor or stem cells are administered once weekly every two weeks. In another example, four doses of mesenchymal lineage precursor or stem cells are administered over two weeks before subsequent doses are administered monthly. In an example, two doses of mesenchymal lineage precursor or stem cells can be administered once weekly every two weeks before subsequent doses are administered once monthly. In an example, four doses are administered monthly.

In an example, compositions of the disclosure comprise a “clinically proven effective” amount of MLPSC. In an example, compositions of the disclosure comprise a “clinically proven effective” amount of MSCs.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### Examples

### Intermediate Stem Cell Populations and Ex-Vivo Culture-Expansion of the Same, for the Treatment of Inflammatory Disease.

### Donor Cell Bank Production and Analysis

Carbon dioxide (CO₂) in the culture medium of culture expanded mesenchymal stem cells (ce-MSC) is required to maintain the buffer between carbonic acid and bicarbonate to ensure that the culture medium is maintained at a physiological pH (7.2-7.4) necessary for optimal cell growth.

Donor cell bank (DCB) comprising ce-MSC were previously produced by culture expanding MSC populations in ten-stack cell factories (CF10) with active priming of 10±2% CO₂. However, when CO₂ concentrations are too high, intracellular pH can be excessively lowered, adversely affecting growth rates and altering the metabolism, productivity, function and glycosylation profiles of mammalian cells. Accordingly, DCB manufacturing methods were changed to require seeding MSCs into CF10 and culturing cells with passive 5±2% CO₂ priming. In other words, passive 5±2% CO₂ priming (referred to herein as “DCB2” methods) represented a departure from previous manufacturing methods (referred to herein as “DCB1” methods) which involved active priming with 10±2% CO₂.

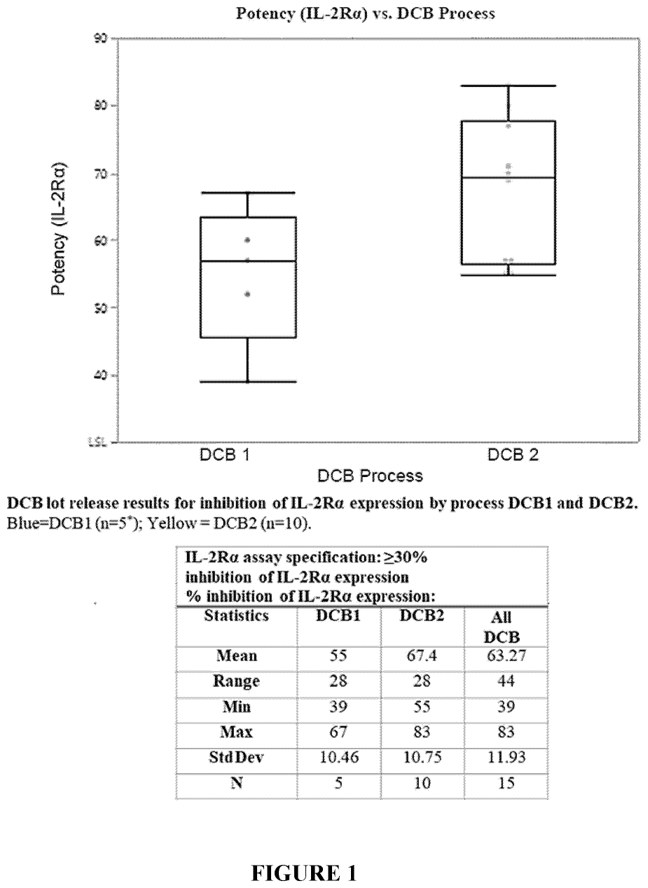

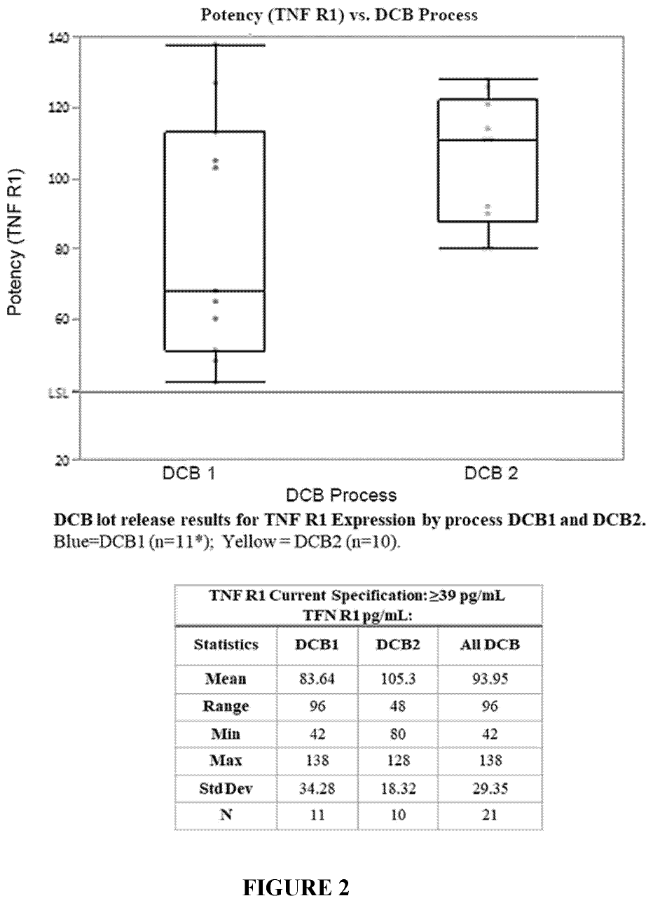

% inhibition of IL2-RA is measured following co-culture of T-cells with the mesenchymal stem cells and inhibition of IL2-RA expression is associated with a suppressive effect on T cell activation, providing an effective measure of therapeutic efficacy (e.g. 100 day survival benefit) in inflammatory diseases such as GvHD. Surprisingly, the present inventors discovered that the cell populations produced using DCB2 methods had increased potency as measured by % IL2-RA inhibition (FIG. 1). Cell populations produced using DCB2 methods also had increased levels of TNFR-1 expression (FIG. 2).

### Donor Product Production and Analysis

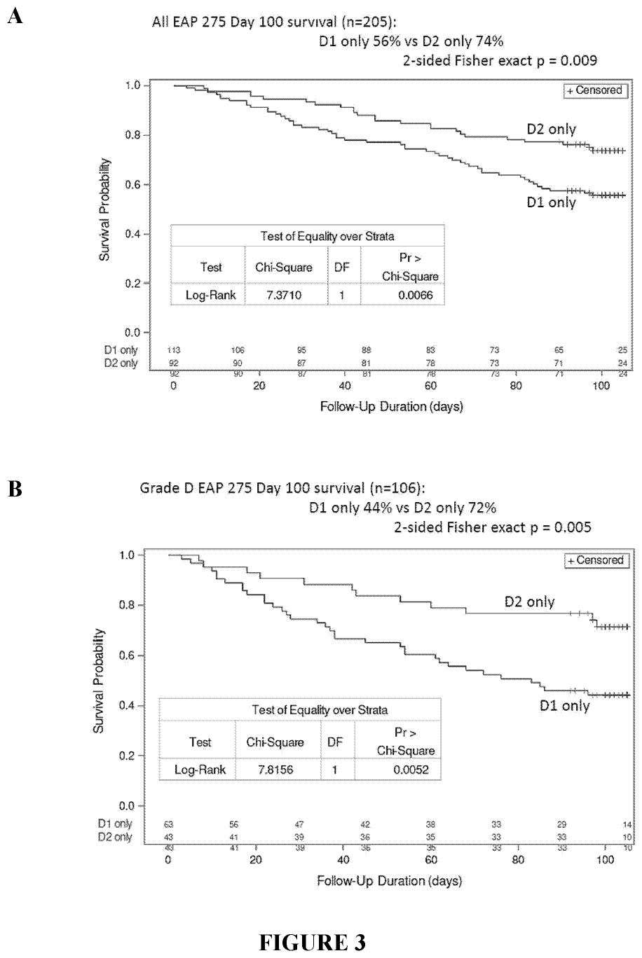

More surprisingly, when DCB1 and DCB2 lots were further expanded to clinical drug product (DP), significantly increased 100 day survival (74%) was observed in patients administered DP derived from DCB2 compared with survival (56%) in patients administered DP derived from DCB1. Increased 100 day survival was further pronounced in patients with severe Grade D disease; 72% in patients administered DP derived from DP2 compared with 44% in patients administered DP derived from DP1. Results shown in Table 1; survival benefit also presented graphically in FIG. 3.

TABLE 1
Improved Survival across all patients with acute GVHD

Day 100 Overall Survival

| | All patients | Grade B/C | Grade D |
|---|---|---|---|
| DCB1 process | 56% | 70% | 44% |
| only (n = 113) | (63) | (35) | (28) |
| DCB2 process | 74% * | 75% | 72% ** |
| only (n = 92) | (68) | (37) | (31) |

* p = 0.006
** p = 0.018

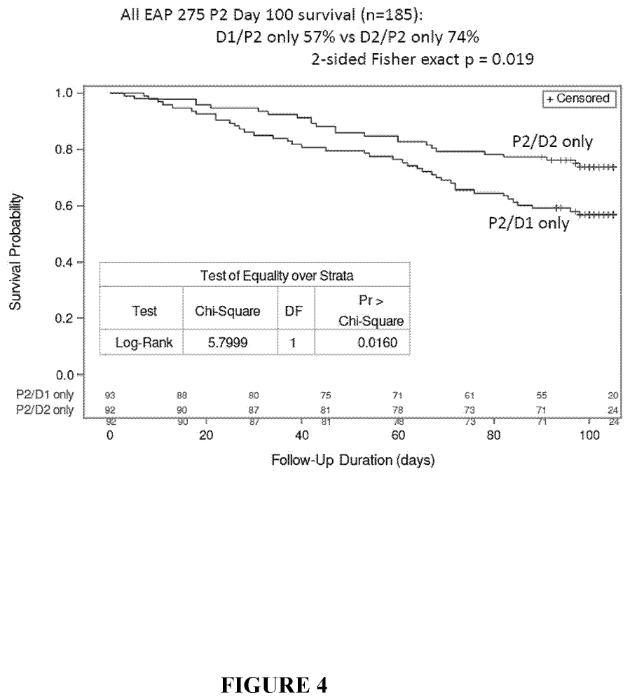

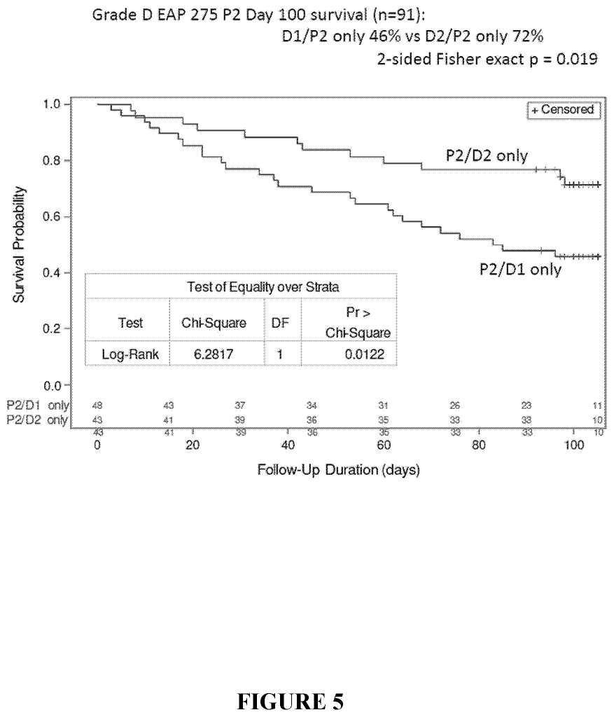

The present inventors then identified that survival benefit could be predicted, and therefore DCB populations could be selected for expansion, based on % IL2-RA inhibition. Interestingly, when DCB1 and DCB2 lots were further expanded to clinical DP, mean % IL2-RA expression was 76.5% in DP derived from DCB2 compared with 63.7% DP derived from DCB1. This is a significant finding because patients administered DP expanded from DCB1 intermediate and characterized by a mean % IL2-RA of 63.7% were associated with 100 day survival of 56%, decreasing to 44% for patients with severe Grade D disease. In contrast, patients administered DP expanded from DCB2 intermediate and characterized by a mean % IL2-RA of 76.5% were associated with 100 day survival of 74%, only decreasing to 72% for patients with severe Grade D disease (FIG. 4; p=0.019). Indeed, survival benefit was most pronounced in patients with severe Grade D disease with 100 day survival of 74% recorded in patients administered DP expanded from DCB2 intermediate reducing to 46% in patients administered DP expanded from DCB1 intermediate (FIG. 5; p=0.012).

As DP from both DCB1 and DCB2 were produced in the same way, thereby controlling for any potential for downstream processes to impact on the final DP characteristics, the present inventors findings underpin the importance of characterizing cell intermediates prior to their expansion to DP, in particular in the context of GvHD therapy, as failure to do so can have significant impacts on overall patient survival. The present inventors findings also underpin significantly improved methods of selecting cell intermediates for large scale culture expansion to drug product, reducing the risk of expending resources expanding cell populations that will not meet necessary potency criteria.

### SUMMARY

Donor Cell Bank manufacturing process provides “memory” for subsequent final product derived from that DCB. Changes in DCB manufacture are associated with higher % IL2-RA inhibition in both DCB and DP. Increased % IL2-RA inhibition are accompanied by significant improvements in survival, particularly in Grade D patients. These findings underpin methods of selecting and analyzing DCBs for use in therapy, in particular treatment of inflammatory disorders such as GvHD.

### Inhibition of IL-2RA on Peripheral Blood Mononuclear Cells (PBMCs) by Mesenchymal Lineage or Precursor Stem Cells (MLPSCs) Preparation of MLPSCs

Cryopreserved human MLPSCs are thawed and resuspended at a concentration of 4.00E+05 viable MLPSCs/mL in a IL-2Ra MLPSCs Culture Medium. The viability assessment is performed using tryptan blue. MLPSCs are then seeded into a 24-well plate. 500 μL of the 4.00E+05 viable MLPSCs/mL is added to each well. Cells are incubated at 37° C.±2° C., 5%±2% CO2 for no more than 24±1 hours. Prior to co-culture with PMBCs, cells are checked for adherence to the plate and to ensure cell morphology is “fibroblast-like” long and flat.

### Preparation of PMBCs

PMBCs are thawed and resuspended at a concentration of 2.00E+06 viable cells/mL in a Co-Culture Medium (50% IL-2RA MLPSCs Culture Medium and 50% DMEM). The viability assessment is performed using tryptan blue.

### Co-Culture of MLPSCs and PBMCs

Stimulated PMBCs are prepared by adding CD3 and CD28 antibodies (final concentration 4 g/ml) to a portion of the 2.00E+06 viable PMBCs. A portion of the viable PMBCs that are not stimulated with CD3 and CD28 antibodies are kept as a negative control. 500 μL of Stimulated PMBCs are then added to the test wells comprising Ce-MLPSC (either intermediate population or drug product).

Stimulated PMBCs alone are used a positive control and unstimulated PMBCs are used a negative control. 500 μL of the respective control samples and 500 μL of Co-Culture Medium is added to control wells of the culture plate.

The MLPSCs and PMBCs are then cultured in an incubator at 37° C.±2° C., 5%±2% CO2 for 72 to 74 hours. Following incubation, PMBC samples are gently harvested, centrifuged, and resuspended in a lysis buffer to prepare cell lysates. Lysates are then incubated on wet ice for 15 minutes.

For example, Ce-MSC (either intermediate population or drug product) are co-cultured with un-stimulated or stimulated (CD3; CD28 antibodies) peripheral blood mononuclear cells (PBMC) as shown below. These conditions ware representative of a ratio of approximately 1 stem cell: 5 PMBC:

| | 1 | 2 | 3 | 4 | 5 | 6 |
|---|---|---|---|---|---|---|
| A | | | | | Sample 1: _{——}_{——}_{——} | |
| B | | 500 μl | | 500 μl | Co-Culture - 500 μl | |
| | | unstimulated | | stimulated | stimulated PBMC + | |
| | | PBMC + 500 | | PBMC + 500 | 500 μl ceMSCs | |
| C | | μl Co- | | μl Co- | □ N/A | |
| | | Culture media | | Culture media | Sample 2: _{——}_{——}_{——} | |
| D | | | | | Co-Culture - 500 μl | |
| | | | | | stimulated PBMC + | |
| | | | | | 500 μl ceMSCs | |

The plate was incubated at 37° C. for 60-84 hours before cells were harvested, lysed and assessed for IL2-RA level using a commercially available ELISA kit, according to the manufacturer's instructions (R&D systems).

### IL-2RA ELISA

IL-2RA standards of 2500 to 78 pg/ml are prepared and added to the ELISA plate along with controls and co-culture lysate samples. An anti-IL-2RA antibody conjugated to HRP is then added to all wells. The plate is incubated for approximately 3 hours at ambient temperature on an orbital shaker with gentle shaking at 200 rpm. Following incubation, the substrate is added to each well. The plate is incubated again in the darkness for 20 minutes at ambient temperature before a Stop solution is added to stop the reaction. Within 30 minutes, the absorbance of wells is read at 450 nm with wavelength correction at 570 nm.

To calculate the amount of IL-2RA, the A450 nm-570 nm value for each well is calculated using standard analysis software (e.g. BioTek). The mean absorbance of the 0 pg/mL negative control Is subtracted from the A450 nm-570 nm value. The concentration of IL-2RA in test samples is interpolated from the standard curve of the IL-R2A standards. The % inhibition of IL-2RA on PBMCs by MLPSCs is calculated using the following formula: % inhibition=[[1−(Co-Culture MLPSCs PBMC/Stimulated PBMC)]×100].

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from AU2022901890 filed 5 Jul. 2022, the disclosures of which are incorporated herein by reference.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of selecting a cell population for culture expansion, the method comprising: (i) obtaining a population of mesenchymal precursor lineage or stem cells (MLPSCs); (ii) culturing the MLPSC population in a culture medium; (iii) determining under culture conditions the level of IL2-RA inhibition; and, (iv) selecting an MLPSC population for further expansion that inhibits IL2-RA by at least 55% under culture conditions.

2. The method of claim 1, which comprises selecting an MLPSC population for further expansion that inhibits IL2-RA by at least 60% under culture conditions.

3. The method of claim 1 or claim 2, wherein the MLPSC population that is selected for further expansion also expresses at least 80 pg/ml TNF-R1 under culture conditions.

4. The method according to any one of claims 1 to 3, further comprising culture expanding a selected MLPSC population to provide a pharmaceutical composition.

5. The method according to any one of claims 1 to 4, wherein culture expansion of the selected MLPSC population provides a MLPSC population characterized by IL2-RA inhibition greater than 60%, greater than 70% under culture conditions.

6. A method for determining therapeutic efficacy of an intermediate population of mesenchymal precursor lineage or stem cells (MLPSCs): (i) obtaining a population of MLPSCs; (ii) culturing the cells in a culture medium; and (iii) determining under culture conditions the level of IL2-RA inhibition, wherein at least 55% IL2-RA inhibition under culture conditions is indicative of therapeutic efficacy.

7. The method of claim 6, wherein IL2-RA inhibition greater than 60%, greater than 70% under culture conditions is indicative of therapeutic efficacy.

8. The method of claim 6 or claim 7, which further comprises determining under culture conditions the level of TNF-R1 expression, wherein expression of at least 80 pg/ml TNF-R1 under culture conditions is indicative of therapeutic efficacy.

9. The method according to any one of claims 6 to 8, wherein upon determining therapeutic efficacy, the intermediate population of MLPSCs is culture expanded to provide a pharmaceutical composition.

10. The method of claim 9, wherein the pharmaceutical composition comprises a MLPSC population characterized by IL2-RA inhibition between 60% and 80% under culture conditions.

11. A method of treating a subject with an inflammatory disease, the method comprising administering to a subject in need thereof a composition comprising a culture expanded population of mesenchymal lineage precursor or stem cells (MLPSCs), wherein the MLPSCs are culture expanded from a population of MLPSCs which inhibit IL2-RA by at least 55% under culture conditions.

12. The method of claim 11, wherein the culture expanded MLPSCs which comprise the administered composition inhibit IL2-RA by at least 56%, at least 60% under culture conditions.

13. The method of claim 11 or claim 12, wherein the MLPSCs are culture expanded from a population of MLPSCs that also express at least 80 pg/ml TNF-R1 under culture conditions.

14. A cryopreserved cellular intermediate comprising a population of culture expanded mesenchymal lineage precursor or stem cells (MLPSC), wherein the population of MLPSCs are culture expanded between 2 and 5 passages and the culture expanded MLPSCs: a) inhibit IL2-RA by at least 55% under culture conditions; and/or b) express at least 80 pg/ml TNF-R1 under culture conditions.

15. The method according to any one of claims 11 to 13 or the cellular intermediate of claim 14, wherein the MLPSCs: a) inhibit IL2-RA by at least 60%, at least 70% under culture conditions; and/or b) expresses at least 100 pg/ml TNF-R1, at least 105 pg/ml TNF-R1 under culture conditions.

16. The method or the cellular intermediate according to any one of claims 1 to 15, wherein the MLPSCs are culture expanded in 2D culture.

17. The method or the cellular intermediate according to any one of claims 1 to 16, wherein the MLPSCs are culture expanded in 5%+/−2% Co₂.

18. The method or the cellular intermediate according to claim 17, wherein the MLPSCs are culture expanded in 5%+/−2% CO₂ with passive priming.

19. The method according to any one of claims 11 to 13, wherein the inflammatory disease is graft versus host disease (GvHD).

20. The method of claim 19, wherein the GvHD is Grade D.

21. The method of claim 19, wherein the GvHD is chronic GvHD.

22. The method according to any one of claims 11 to 21, wherein treatment increases 100 day survival to greater than 60%, greater than 70%.

23. The method according to any one of claims 11 to 22, wherein the subject is refractory to steroid immunosuppressant and/or a biologic therapy.

24. The method according to any one of claims 11 to 23, wherein the subject receives at least two doses of the composition.

25. The method or the cellular intermediate according to any one of claims 1 to 24, wherein the MLPSCs are mesenchymal stem cells.

26. A method of manufacturing drug product which comprises a population of mesenchymal lineage precursor or stem cells (MLPSCs), the method comprising: acquiring a determination of whether a test population of MLPSCs have a predetermined level of IL2-RA inhibition under culture conditions, and culture expanding at least a portion of the test population of MLPSCs to a drug product if the test population of MLPSCs have at least the predetermined level of IL2-RA inhibition under culture conditions, thereby manufacturing the drug product; or discarding at least a portion of the test population of MLPSCs if the population of MLPSCs has less than the predetermined level of IL2-RA inhibition under culture conditions.

27. The method of claim 26, wherein the predetermined level of IL2-RA inhibition is at least 55% under culture conditions.

28. The method of claim 26, wherein the predetermined level of IL2-RA inhibition is at least 60% under culture conditions.

29. The method according to any one of claims 26 to 28, the method further comprising acquiring a determination of whether a test population of MLPSCs have a predetermined level of TNF-R1 under culture conditions, and culture expanding at least a portion of the test population of MLPSCs to a drug product if the test population of MLPSCs have at least the predetermined level of IL2-RA inhibition under culture conditions and the predetermined level of TNF-R1 under culture conditions, thereby manufacturing the drug product; or discarding at least a portion of the test population of MLPSCs if the population of MLPSCs has less than the predetermined level of IL2-RA inhibition under culture conditions and less than the predetermined level of TNF-R1 under culture conditions.

30. The method of claim 29, wherein the predetermined level of TNF-R1 is at least 80 pg/ml, at least 100 pg/ml, at least 105 pg/ml.

31. The method according to any one of claims 26 to 30, wherein culture expanding at least a portion of the test population of MLPSCs to a drug product provides a MLPSC population characterized by IL2-RA inhibition greater than 60%, greater than 70% under culture conditions.
